(19) United States
(12) Patent Application Publication
(10) Pub. No.: US 2025/0225564 A1
(43) Pub. Date: Jul. 10, 2025

(54) SKIN CARE SYSTEM AND SKIN CARE METHOD THEREOF

(71) Applicant: LG HOUSEHOLD & HEALTH CARE LTD., Seoul (KR)

(72) Inventors: Hae Young HWANG, Seoul (KR); Bok Hyun PACK, Seoul (KR); Sung Tai PARK, Seoul (KR)

(73) Assignee: LG HOUSEHOLD & HEALTH CARE LTD., Seoul (KR)

(21) Appl. No.: 19/009,233

(22) Filed: Jan. 3, 2025

(30) Foreign Application Priority Data
Jan. 5, 2024 (KR) 10-2024-0002367

(51) Int. Cl. G06Q 30/0601; G16H 10/20

(52) U.S. Cl. CPC G06Q 30/0631; G06Q 30/0621; G16H 10/20

(57) ABSTRACT

A skin care system according to an embodiment of the present invention includes a mobile terminal, a dermatology hospital terminal, and a skin care shop terminal, and provides customized skin treatment and skin management to a user through linked operations among a mobile terminal, a dermatology hospital terminal, and a skin care shop terminal.

### BACKGROUND OF THE INVENTION

### 1. Field of the Invention

The present invention relates to a skin care system.

### 2. Discussion of the Related Art

In recent decades, interest in skin care has increased to an unprecedented level along with the rapid growth of the beauty industry. This goes beyond a simple matter of appearance and has a great impact on an individual's quality of life and self-development.

In the past, users visited dermatology clinics to receive skin care/procedure/prescriptions. In addition, users went to skin care shops to receive skin care based on the prescriptions they received from dermatology clinics.

However, in the past, there was no exchange of information between dermatology clinics and skin care shops, making it difficult to perform accurate post-treatment care at skin care shops.

In addition, even if post-treatment care was received at a skin care shop, additional treatment may be required, but appropriate measures were not taken for this.

### SUMMARY OF THE INVENTION

The purpose of the present invention is to provide customized skin treatment and skin management to a user through linked operations among a mobile terminal, a dermatology hospital terminal, and a skin care shop terminal.

A skin care system including a mobile terminal, a dermatology hospital terminal, and a skin care shop terminal according to an embodiment of the present invention, the dermatology hospital terminal is configured to receive user information from the mobile terminal, acquire first skin condition information indicating a user's skin condition corresponding to the received user information, generate first treatment information and first skin post-treatment management information according to a skin treatment performed based on the first skin condition information, and transmit the first treatment information and the first skin post-treatment management information to the skin care shop terminal, and the skin care shop terminal is configured to perform post-treatment management according to the first skin post-treatment management information, acquire skin care status information including second skin condition information of the user according to a result of performing the post-treatment management, and transmit the skin care status information to the dermatology hospital terminal, and the dermatology hospital terminal is configured to determine whether additional skin care of the user is necessary based on the skin care status information.

According to an embodiment of the present invention, effective customized skin diagnosis/treatment/management can be made possible by integrated management of the user's skin through linked operations among the user's mobile terminal, dermatology hospital terminal, and skin care shop terminal.

### BRIEF DESCRIPTION OF THE DRAWINGS

FIG. 1 is a drawing explaining the configuration of a skin care system according to an embodiment of the present invention.

FIG. 2 is a block diagram explaining the configuration of a mobile terminal according to an embodiment of the present invention.

FIG. 3 is a block diagram explaining the configuration of a dermatology hospital terminal according to an embodiment of the present invention.

FIG. 4 is a block diagram explaining the configuration of a skin care shop terminal according to an embodiment of the present invention.

FIG. 5 is a ladder diagram explaining a skin care method of a skin care system according to an embodiment of the present invention.

FIGS. 6A and 6B are drawings explaining an example of a skin care system according to another embodiment of the present invention.

FIG. 7 is a drawing explaining a method of generating post-processing management information according to treatment information according to an embodiment of the present invention.

### DETAILED DESCRIPTION OF THE PREFERRED EMBODIMENTS

Hereinafter, embodiments of the present invention will be described in detail with reference to the drawings. The suffixes “module”, “unit” or “portion” for components used in the following description are merely provided only for facilitation of preparing this specification, and thus they are not granted a specific meaning or function.

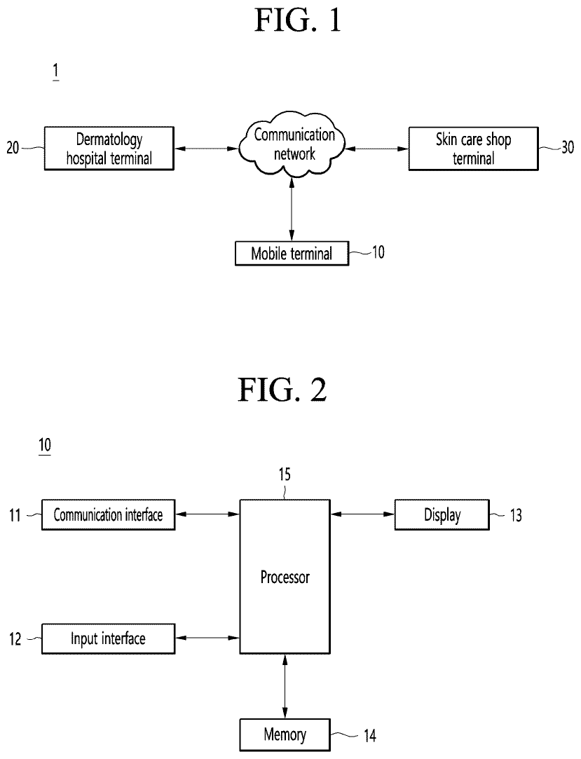

FIG. 1 is a drawing explaining the configuration of a skin care system according to an embodiment of the present invention.

Referring to FIG. 1, the skin care system may include a mobile terminal 10, a dermatology hospital terminal 20, and a skin care shop terminal 30.

The mobile terminal 10, the dermatology hospital terminal 20, and the skin care shop terminal 30 may communicate with each other through a communication network. The communication network may be an Internet communication network, but this is only an example.

The mobile terminal 10 may obtain user information.

The mobile terminal 10 may transmit the obtained user information to the dermatology hospital terminal 20.

The dermatology hospital terminal 20 may obtain first skin condition information indicating the skin condition of the user corresponding to the user information.

The dermatology hospital terminal 20 may generate first skin post-treatment management information according to the skin treatment performed based on the first skin condition information.

The dermatology hospital terminal 20 can transmit user information, first treatment information of a doctor, and the first skin post-treatment management information to the skin care shop terminal 30.

The skin care shop terminal 30 can perform post-treatment management on the user's skin based on the received first skin post-treatment management information.

The skin care shop terminal 30 can obtain skin care status information of the user according to the performance of skin post-treatment management.

The skin care shop terminal 30 can transmit skin care status information of the user to the dermatology hospital terminal 20.

The dermatology hospital terminal 20 can determine whether additional skin care is necessary based on the received skin care status information of the user.

If the dermatology hospital terminal 20 determines that additional skin care is necessary, the dermatology hospital terminal 20 can transmit an additional skin care notification to the mobile terminal 10.

The mobile terminal 10 can display the received additional skin care notification on the display 13.

The mobile terminal 10 can transmit an additional skin care request to the dermatology hospital terminal 20 through the communication interface 11.

The dermatology hospital terminal 20 can generate second treatment information of the doctor and second skin post-treatment management information according to the additional skin care request.

FIG. 2 is a block diagram illustrating the configuration of a mobile terminal according to an embodiment of the present invention.

The mobile terminal 10 may be a portable terminal such as a user's smart phone, smart pad, etc.

Referring to FIG. 2, the mobile terminal 10 may include a communication interface 11, an input interface 12, a display 13, a memory 14, and a processor 15.

The communication interface 11 may transmit and receive data with external devices using wired/wireless communication technology.

The communication interface 11 may transmit and receive sensor information, user input, learning models, control signals, etc. with external devices.

The communication technologies used by the communication interface 11 include GSM (Global System for Mobile communication), CDMA (Code Division Multi Access), LTE (Long Term Evolution), 5G, WLAN (Wireless LAN), Wi-Fi (Wireless-Fidelity), etc.

The input interface 12 may include a camera for inputting a video signal, a microphone for receiving an audio signal, a user input module for receiving information from a user, etc. The camera or microphone may be treated as a sensor, and a signal obtained from the camera or microphone may be referred to as sensing data or sensor information.

The display 13 may display information processed in the mobile terminal 10. The display 13 may display execution screen information of an application running in the mobile terminal 10, or UI (User Interface) or GUI (Graphical User Interface) information according to such execution screen information.

The display 13 can implement a touch screen by forming a mutual layer structure with the touch sensor or forming it as an integral part. The touch screen can provide the function of an input interface between the mobile terminal 10 and the user, and at the same time, can provide an output interface between the mobile terminal 10 and the user.

The memory 14 can store information processed in the mobile terminal 10.

The processor 15 can control the overall operation of the mobile terminal 10. The specific operation of the processor 15 will be described later.

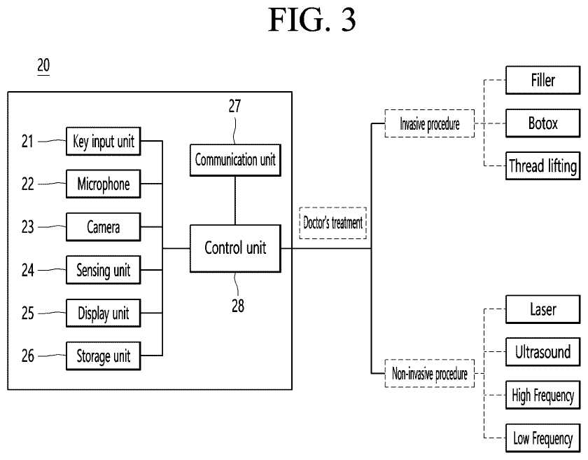

FIG. 3 is a block diagram illustrating the configuration of a dermatology hospital terminal according to an embodiment of the present invention.

The dermatology hospital terminal 20 can include a key input unit 21, a microphone 22, a camera 23, a sensing unit 24, a display unit 25, a storage unit 26, a communication unit 27, and a control unit 28.

The dermatology hospital terminal 20 can be any one of a computer, a laptop, a PC, or a skin treatment device, but is only an example.

The key input unit 21 can have multiple keys and can obtain key inputs from a doctor or nurse.

The microphone 22 can receive a voice signal. The microphone 22 can receive a voice signal corresponding to a voice command spoken by a user.

The camera 23 can capture image data. The camera 23 can capture image data for a specific body part of the user.

The sensing unit 24 can sense skin data for the user's skin. The sensing unit 24 can sense the user's skin condition. The sensing unit 24 can include a moisture sensor, a temperature sensor, an optical sensor, an infrared sensor, etc.

The display unit 25 can display information processed in the dermatology hospital terminal 20.

The storage unit 26 can store information processed in the dermatology hospital terminal 20.

The communication unit 27 can transmit and receive data with external devices using wired/wireless communication technology. The communication technologies used by the communication unit 27 include GSM (Global System for Mobile communication), CDMA (Code Division Multi Access), LTE (Long Term Evolution), 5G, WLAN (Wireless LAN), and Wi-Fi (Wireless-Fidelity).

The control unit 28 can control the overall operation of the dermatology hospital terminal 20.

The specific operation of the control unit 28 is described later.

The dermatology hospital terminal 20 can be placed in a dermatology hospital. A dermatologist can perform either an invasive procedure or a non-invasive procedure on a user.

The invasive procedure can include fillers, botox, and thread lifting.

The non-invasive procedures may include laser procedures, ultrasound procedures, high-frequency procedures, and low-frequency procedures.

A dermatology hospital may be equipped with multiple invasive procedure devices for invasive procedures and multiple non-invasive procedure devices for non-invasive procedures.

The multiple invasive procedure devices and multiple non-invasive procedure devices may be included in the dermatology hospital terminal 20 or may be equipped separately.

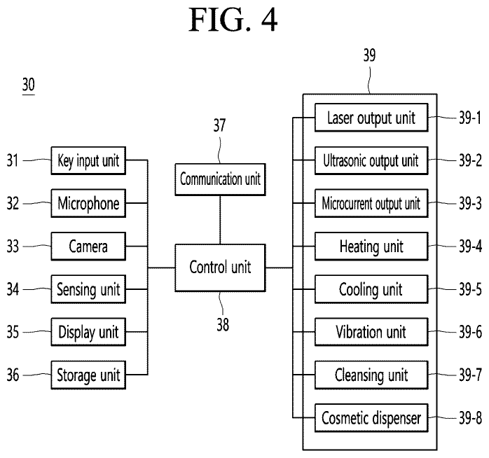

FIG. 4 is a block diagram illustrating a configuration of a skin care shop terminal according to an embodiment of the present invention.

The skin care shop terminal 30 may be any one of a computer, a laptop, a PC, and a skin care machine, but is only an example.

The skin care shop terminal 30 may include a key input unit 31, a microphone 32, a camera 33, a sensing unit 34, a display unit 35, a storage unit 36, a communication unit 37, a control unit 38, and a skin care module 39.

The key input unit 31 may have multiple keys and may obtain key input from a skin care professional or manager.

The microphone 32 may receive a voice signal. The microphone 32 may receive a voice signal corresponding to a voice command spoken by the user.

The camera 33 may capture image data. The camera 33 may capture image data for a specific body part of the user.

The sensing unit 34 may sense skin data for the user's skin. The sensing unit 34 may sense the user's skin condition. The sensing unit 34 may include a moisture sensor, a temperature sensor, an optical sensor, an infrared sensor, and the like.

The display unit 35 can display information processed in the skin care shop terminal 30.

The storage unit 36 can store information processed in the skin care shop terminal 30.

The communication unit 37 can transmit and receive data with external devices using wired/wireless communication technology. The communication technologies used by the communication unit 27 include GSM (Global System for Mobile communication), CDMA (Code Division Multi Access), LTE (Long Term Evolution), 5G, WLAN (Wireless LAN), Wi-Fi (Wireless-Fidelity), etc.

The control unit 38 can control the overall operation of the skin care shop terminal 30.

The specific operation of the control unit 38 will be described later.

The skin care module 39 may include a laser output unit 39-1, an ultrasonic output unit 39-2, a microcurrent output unit 39-3, a heating unit 39-4, a cooling unit 39-5, a vibration unit 39-6, a cleansing unit 39-7, and a cosmetic dispenser 39-8.

The laser output unit 39-1 may irradiate a laser to the user's skin.

The ultrasonic output unit 39-2 may output ultrasonic waves to the user's skin.

The microcurrent output unit 39-3 may output microcurrent to the user's skin.

The heating unit 39-4 can apply heat of a specific temperature to the user's skin.

The cooling unit 39-5 can cool the user's skin.

The vibration unit 39-6 can apply a vibration signal to the user's skin.

The cleansing unit 39-7 can cleanse the user's skin.

The cosmetic dispenser 39-8 can discharge a certain amount of cosmetics. The cosmetic dispenser 39-8 can discharge a certain amount of each of a plurality of cosmetics.

The cosmetic dispenser 39-8 can mix a plurality of cosmetics according to the cosmetic mixing ratio included in the skin post-treatment management information under the control of the control unit 38, and then discharge the mixed cosmetics.

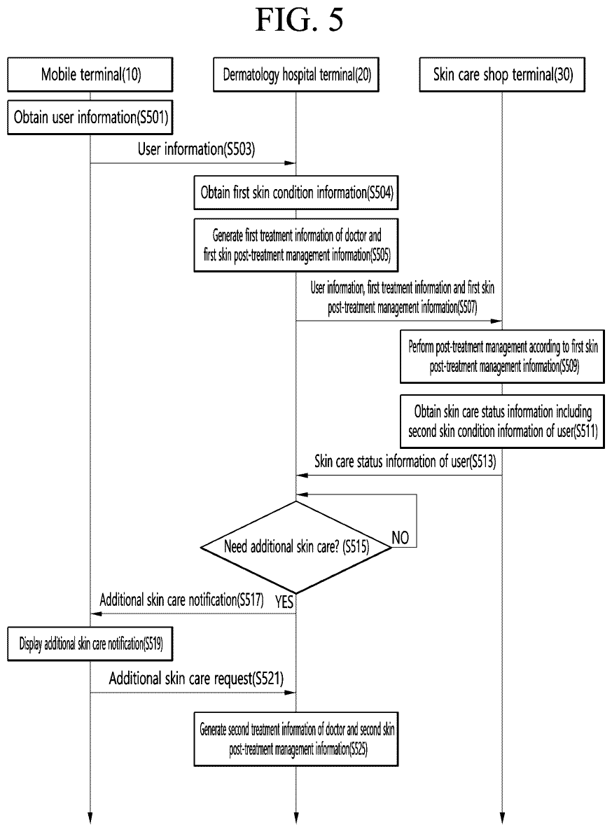

FIG. 5 is a ladder diagram for explaining a skin care method of a skin care system according to an embodiment of the present invention.

Referring to FIG. 5, the processor 15 of the mobile terminal 10 can obtain user information (S501).

The user information can include user identification information and basic information. The user identification information is information that can identify the user, and can include at least one of the user's name and the user's account.

The user's basic information can include the user's name, age, gender, etc.

The processor 15 of the mobile terminal 10 can receive user information by executing a skin care application installed on the mobile terminal 10.

The processor 15 of the mobile terminal 10 can transmit the obtained user information to the dermatology hospital terminal 20 through the communication interface 11 (S503).

The control unit 28 of the dermatology hospital terminal 20 can obtain the first skin condition information indicating the skin condition of the user corresponding to the user information (S504).

In one embodiment, the control unit 28 of the dermatology hospital terminal 20 can receive first skin condition information from the mobile terminal 10. The mobile terminal 10 can collect first skin condition information related to the skin condition of the user through a skin care application, and transmit the collected first skin condition information to the dermatology hospital terminal 20.

In another embodiment, the control unit 28 of the dermatology hospital terminal 20 can independently obtain first skin condition information. The control unit 28 can obtain first skin condition information through skin information input by a doctor through the key input unit 21 or sensing information sensed through the sensing unit 24.

The first skin condition information can include one or more of the questionnaire results of the user's skin, the results of an ON/OFF line skin consultation, and skin sensing information.

The first skin condition information can include one or more skin condition factors. One or more skin condition factors may include information on skin oil and moisture, skin brightness, skin pigmentation, skin response to stimulation, and skin elasticity.

The control unit 28 can match the user information and the first skin condition information and store them in the storage unit 26.

The control unit 28 of the dermatology hospital terminal 20 can generate the first treatment information and the first skin post-treatment management information according to the skin treatment performed based on the first skin condition information (S505).

The doctor can perform one or more of an invasive procedure or a non-invasive procedure on the user's skin based on the first skin condition information.

The control unit 28 of the dermatology hospital terminal 20 can obtain the first treatment information, which is information on the performed procedure. The first treatment information can be generated by the doctor's input through the key input unit 21.

The first treatment information can include information on the type of the procedure, the method of the procedure, the number of procedures, and the schedule of the procedure.

The type of the procedure can include an invasive procedure and a non-invasive procedure.

The method of the procedure may include fillers, botox, and thread lifting in the case of invasive procedure, and may include laser treatment, ultrasound treatment, radiofrequency treatment, and low-frequency treatment in the case of non-invasive procedure.

The control unit 28 of the dermatology hospital terminal 20 may generate first skin post-treatment management information based on the first treatment information.

The storage unit 26 can store skin post-treatment management information matched to the treatment information.

The control unit 28 can extract the first skin post-treatment management information matched to the first treatment information through the storage unit 26.

The first skin post-treatment management information can include at least one of a post-treatment management method or customized cosmetic information.

The post-treatment management method can be a method for performing at least one of low-stimulation management, cold management, heat management, moisture management, massage, steaming, cosmetic application, cupping, meridian massage, and moisture pack management.

The customized cosmetic information can include at least one of a type of cosmetic and a mixing ratio of the cosmetic. The type of cosmetic can be any one of a basic cosmetic, a pack, a sunscreen, and a cover foundation.

In one embodiment, the control unit 28 may match the first treatment information and the first skin post-treatment management information with the user information and store them in the storage unit 26.

In another embodiment, the control unit 28 may match the first treatment information and the first skin post-treatment management information with the user information and store them in a separate external database (not shown).

Meanwhile, the post-treatment management method may vary depending on the treatment information. This will be described later.

The control unit 28 of the dermatology hospital terminal 20 can transmit user information, the first treatment information of the user, and the first skin post-treatment management information to the skin care shop terminal 30 through the communication unit 27 (S507).

The control unit 38 of the skin care shop terminal 30 can perform post-treatment management on the user's skin based on the received first skin post-treatment management information (S509).

The control unit 38 can control the skin care module 39 based on the post-treatment management method and customized cosmetic information included in the first skin post-treatment management information.

The control unit 38 can perform post-treatment management on the user's skin according to the control of the skin care module 39.

The control unit 38 can control the operation of the laser output unit 39-1, the ultrasonic output unit 39-2, the microcurrent output unit 39-3, the heating unit 39-4, the cooling unit 39-5, and the vibration unit 39-6 included in the skin care module 39 according to the post-treatment management method.

The control unit 38 can control the operation of the cleansing unit 39-7 and the cosmetic dispenser 39-8 included in the skin care module 39 according to the customized cosmetic information.

The control unit 38 of the skin care shop terminal 30 can obtain skin care status information of the user according to the performance of the skin post-treatment management (S511).

The skin care status information may include the management status of the user's skin performed by the skin care module 39 of the skin care shop terminal 30.

The skin care status information may include the management status of the user's skin by schedule.

The skin care status information may include the second skin condition information and post-treatment performance information of the user.

The second skin condition information may include one or more of skin oil and moisture information, skin brightness, skin pigmentation, skin stimulation response, and skin elasticity information.

The post-treatment performance information may include the performance result of the post-treatment management method and the cosmetic administration result according to the customized cosmetic information.

The control unit 38 of the skin care shop terminal 30 may transmit the skin care status information of the user to the dermatology hospital terminal 20 through the communication unit 37 (S513).

The control unit 28 of the dermatology hospital terminal 20 can determine whether additional skin care is necessary based on the received skin care status information of the user (S515).

The control unit 28 can determine whether additional skin care is necessary by comparing the second skin condition information and the first skin condition information included in the skin care status.

If the difference between the value of the skin condition factor included in the second skin condition information and the value of the skin condition factor included in the first skin condition information is less than a certain value, the control unit 28 can determine that additional skin care is necessary.

If the difference between the value of the skin condition factor included in the second skin condition information and the value of the skin condition factor included in the first skin condition information is greater than the certain value, the control unit 28 can determine that additional skin care is unnecessary.

For example, if the difference between the second elasticity level of the skin included in the second skin condition information and the first elasticity level of the skin included in the first skin condition information is less than a certain value, the control unit 28 can determine that additional skin care is necessary.

If the difference between the second elasticity level of the skin included in the second skin condition information and the first elasticity level of the skin included in the first skin condition information is greater than the certain value, the control unit 28 can determine that additional skin care is unnecessary.

In another embodiment, the control unit 28 can determine whether additional skin care is necessary by comparing the first skin post-treatment management information with the post-treatment performance information.

If the control unit 28 determines that, based on the comparison result, the post-treatment is not performed according to the first skin post-treatment management information, the control unit 28 may determine that additional skin care is required.

If the control unit 28 of the dermatology hospital terminal 20 determines that additional skin care is required through the communication unit 27, the control unit 28 may transmit an additional skin care notification to the mobile terminal 10 (S517).

The additional skin care notification may be a notification indicating that additional treatment or additional skin care is required for the user's skin.

The additional skin care notification may be a notification indicating the need for an additional questionnaire or additional offline consultation for the user's skin for the additional treatment or additional skin care.

The dermatology hospital terminal 20 may perform a non-face-to-face treatment with the user through a skin care application.

The additional skin care notification may be a request for a non-face-to-face treatment through the skin care application.

The processor 15 of the mobile terminal 10 can display the received additional skin care notification on the display 13 (S519).

The processor 15 can display the additional skin care notification on the display 13 through the skin care application.

The processor 15 of the mobile terminal 10 can transmit the additional skin care request to the dermatology hospital terminal 20 through the communication interface 11 (S521).

The additional skin care request may include one or more of additional questionnaire information about the user's skin and dermatology hospital reservation information.

The processor 15 may receive input of additional questionnaire information about the user's skin through the skin care application, and transmit the input additional questionnaire information together with the user information to the dermatology hospital terminal 20.

The control unit 28 of the dermatology hospital terminal 20 may generate the second treatment information of the doctor and the second skin post-treatment management information according to the additional skin care request (S525).

The control unit 28 of the dermatology hospital terminal 20 may determine the second treatment information based on the results of a non-face-to-face treatment with the user through the skin care application.

When the doctor's second treatment information and the second skin post-treatment management information are generated, the doctor's prescription can be made through voice recognition, text conversion, management status information, and additional skin care request information so that the user and the doctor can have an online non-face-to-face treatment and asynchronous (e.g., advance reservation) generation.

The second treatment information can include information about additional treatments to be performed on the user.

The second treatment information can include information about the type of treatment, the method of the treatment, the number of treatments, and the treatment schedule.

The second skin post-treatment management information can include one or more of a post-treatment management method or customized cosmetic information for additional post-processing based on the second treatment information.

In one embodiment, the control unit 28 may match the second treatment information and the second skin post-treatment management information with the user information and store them in the storage unit 26.

In another embodiment, the control unit 28 may match the second treatment information and the second skin post-treatment management information with the user information and store them in a separate external database.

Thereafter, the skin care system 1 may repeatedly perform the steps S507 and subsequent processes.

In this way, according to an embodiment of the present invention, effective customized skin diagnosis/treatment/management may be possible by integratedly managing the user's skin through linked operations between the user's mobile terminal 10, the dermatology hospital terminal 20, and the skin care shop terminal 30.

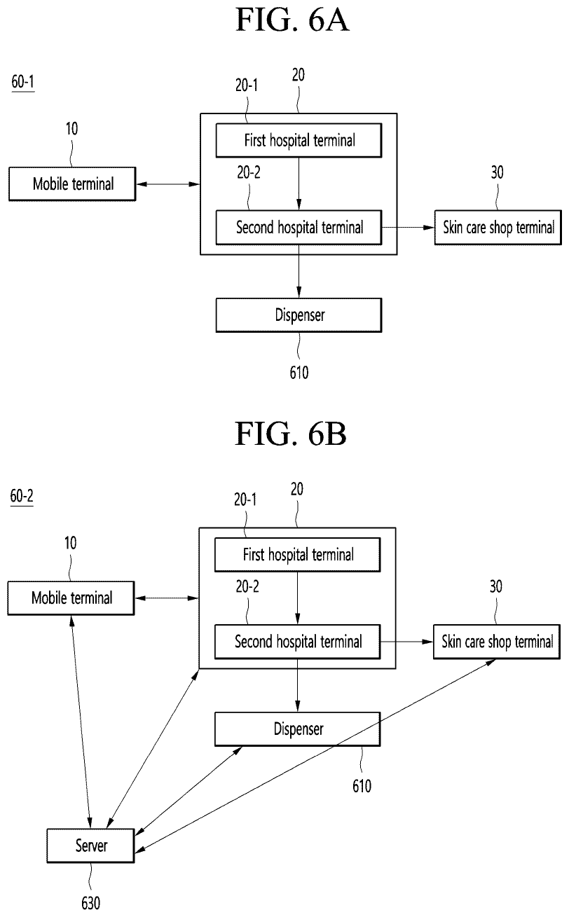

FIG. 6A and FIG. 6B are drawings illustrating an example of a skin care system according to another embodiment of the present invention.

The skin care system 60-1 may include a mobile terminal 10, a dermatology hospital terminal 20, a skin care shop terminal 30, and a dispenser 610.

The dispenser 610 may be installed in one or more of a dermatology hospital or a skin care shop.

The dermatology hospital terminal 20 may include a first hospital terminal 20-1 and a second hospital terminal 20-2.

The first hospital terminal 20-1 may obtain information on a doctor's diagnosis and prescription. The information on the diagnosis and the prescription may include the type of treatment and the schedule of the treatment.

The second hospital terminal 20-2 may store user information received from the mobile terminal 10 and reserve a dermatology treatment for the user.

The second hospital terminal 20-2 may determine a personalized customized cosmetic formulation based on the information on the prescription. The personalized customized cosmetic formulation may include a mixing ratio of cosmetics

The second hospital terminal 20-2 may determine a skin post-treatment management method based on the information on the prescription. The skin post-treatment management method may include one or more of a massage, a steaming, and a cosmetic application.

The dispenser 610 may dispense cosmetics according to a personalized customized cosmetic formulation determined through the second hospital terminal 20-2.

The skin care shop terminal 30 may be placed within a dermatology hospital or may be placed outside of a dermatology hospital.

The skin care shop terminal 30 may perform post-treatment management of the skin according to the personalized customized cosmetic formulation information received from the second hospital terminal 20-2.

The user can input user information, make a reservation at a dermatology hospital, make a reservation at a skin shop, or purchase cosmetics through the mobile terminal 10.

The mobile terminal 10 can receive a home technology method from the second hospital terminal 20-2.

Referring to FIG. 6B, a skin care system 60-2 according to another embodiment of the present invention can include a mobile terminal 10, a dermatology hospital terminal 20, a skin care shop terminal 30, a dispenser 610, and a server 630.

That is, the skin care system 60-2 can further include a server 630 compared to the components of FIG. 6A.

The server 630 can control the skin care system 60-2 as a whole.

The server 630 can control each component of the skin care system 60-2. The server 630 can exchange information with each component of the skin care system 60-2 through a communication network.

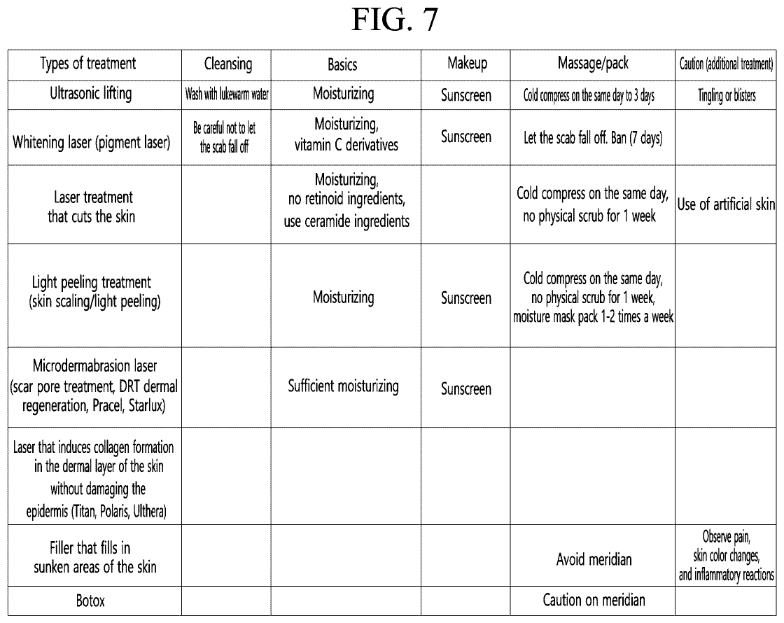

FIG. 7 is a diagram explaining a method of generating post-processing management information according to treatment information according to an embodiment of the present invention.

Referring to FIG. 7, a table 700 including skin post-treatment management methods matched to treatment types is illustrated.

The table 700 can represent post-treatment management methods matched to the doctor's treatment types. The post-treatment management methods can include cleansing items, basic items representing basic cosmetics, makeup items, massage/pack items, and caution items.

The storage unit 26 of the dermatology hospital terminal 20 can store a table 700 including skin post-treatment management methods that match the treatment type.

The control unit 28 of the dermatology hospital terminal 20 can use the table 700 to generate first skin post-treatment management information that matches the first treatment type included in the first treatment information, and can generate second skin post-treatment management information that matches the second treatment type included in the second treatment information.

Each of the first treatment information and the second treatment information can include information on the type of treatment, the method of the treatment, the number of treatments, and the treatment schedule.

For example, if the treatment type is ultrasonic lifting, the post-processing management information can include information on washing with lukewarm water, using moisturizing cosmetics, using sunscreen, cold steaming within 3 days from the same day, and guidance on stinging or blisters.

The control unit 28 of the dermatology hospital terminal 20 can transmit the first skin post-treatment management information or the second skin post-treatment management information generated through the table 700 to the skin care shop terminal 30.

In this way, according to an embodiment of the present invention, the post-treatment management method can be changed according to the treatment information, and as the post-treatment management method is transmitted to the skin care shop terminal 30, more effective and customized skin care can be performed.

According to one embodiment of the present disclosure, the above-described method can be implemented as a processor-readable code on a medium in which a program is recorded. Examples of the processor-readable medium include ROM, RAM, CD-ROM, magnetic tape, floppy disk, optical data storage device, etc.

The skin care system described above is not limited to the configuration and method of the embodiments described above, and the embodiments may be configured by selectively combining all or part of each embodiment so that various modifications can be made.

What is claimed is:

1. A skin care system including a mobile terminal, a dermatology hospital terminal, and a skin care shop terminal,
wherein the dermatology hospital terminal is configured to:
receive user information from the mobile terminal,
acquire first skin condition information indicating a user's skin condition corresponding to the received user information,
generate first treatment information and first skin post-treatment management information according to a skin treatment performed based on the first skin condition information, and
transmit the first treatment information and the first skin post-treatment management information to the skin care shop terminal, and
the skin care shop terminal is configured to:
perform post-treatment management according to the first skin post-treatment management information,
acquire skin care status information including second skin condition information of the user according to a result of performing the post-treatment management, and
transmit the skin care status information to the dermatology hospital terminal, and
the dermatology hospital terminal is configured to:
determine whether additional skin care of the user is necessary based on the skin care status information.

2. The skin care system according to claim 1, wherein the dermatology hospital terminal is configured to:
if it is determined that additional skin care is needed, transmit an additional skin care notification to the mobile terminal.

3. The skin care system according to claim 2, wherein the additional skin care notification is a notification informing the user of the need for additional questionnaire or additional offline consultation related to the user's skin.

4. The skin care system according to claim 3, wherein the mobile terminal is configured to transmit an additional skin care request to the dermatology hospital terminal, and
the additional skin care request includes at least one of additional questionnaire information and dermatology hospital reservation information.

5. The skin care system according to claim 4, wherein the dermatology hospital terminal is configured to:
generate second treatment information including information on additional procedures to be performed on the user according to the additional skin care request and second skin post-treatment management information for additional post-treatment based on the second treatment information.

6. The skin care system according to claim 1, wherein the dermatology hospital terminal is configured to:
determine whether additional skin care is necessary based on a comparison result between the first skin condition information and the second skin condition information.

7. The skin care system according to claim 6, wherein the dermatology hospital terminal is configured to:
determine that additional skin care is necessary if a difference between a value of a skin condition factor included in the second skin condition information and a value of a skin condition factor included in the first skin condition information is less than a certain value.

8. The skin care system according to claim 1, wherein the first skin post-treatment management information includes at least one of a post-treatment management method or customized cosmetic information.

9. The skin care system according to claim 8, wherein the post-treatment management method includes at least one of low-stimulation management, management, heat management, moisture management, massage, steaming, cosmetic application, cupping, meridian massage, and moisture pack management, and
the customized cosmetic information includes at least one of types of cosmetics and a mixing ratio of cosmetics.

10. A skin care method of a skin care system including a mobile terminal, a dermatology hospital terminal, and a skin care shop terminal, comprising:
receiving, by the dermatology hospital terminal, user information from the mobile terminal;
acquiring, by the dermatology hospital terminal, first skin condition information indicating a skin condition of a user corresponding to the received user information;
generating, by the dermatology hospital terminal, first treatment information and first skin post-treatment management information according to skin treatment performed based on the first skin condition information;
transmitting, by the dermatology hospital terminal, the first treatment information and the first skin post-treatment management information to the skin care shop terminal;
performing, by the skin care shop terminal, post-treatment management according to the first skin post-treatment management information;
acquiring, by the skin care shop terminal, skin care status information including second skin condition information of the user according to a result of performing the post-treatment management;
transmitting, by the skin care shop terminal, the skin care status information to the dermatology hospital terminal; and
determining, by the dermatology hospital terminal, whether the user needs additional skin care based on the skin care status information.
